(19) United States
(12) Patent Application Publication
(10) Pub. No.: US 2012/0264776 A1
(43) Pub. Date: Oct. 18, 2012

(54) MENTHOL LIQUIDS COMPOSITION

(76) Inventor: Jitendra Krishan Somani, Waterloo (CA)

(21) Appl. No.: 13/435,054

(22) Filed: Mar. 30, 2012

Related U.S. Application Data
(60) Provisional application No. 61/470,072, filed on Mar. 31, 2011.

(51) Int. Cl. A61K 31/045; A61K 31/485; A61P 11/14

(52) U.S. Cl. 514/289; 514/729

(57) ABSTRACT

A liquid antitussive composition comprising about 0.05 grams to about 0.2 grams of menthol per 100 ml of liquid antitussive composition, a solvent system, and a surfactant, wherein the composition has a maximum alcohol content of about 0.5 grams per 100 ml of the liquid antitussive composition, and wherein the composition is stable for about 6 months at 40° C., 75% relative humidity conditions.

### CROSS REFERENCE TO RELATED APPLICATION

This application claims the benefit of priority from U.S. Provisional Patent Application No. 61/470,072, filed on Mar. 31, 2011, the content of which is incorporated by reference.

### BACKGROUND OF THE INVENTION

1. Field of the Invention

The present invention relates to liquid compositions. More particularly, the present invention relates to menthol liquid compositions and the method of making said compositions.

2. Related Background Art

Cough suppressant medications are readily available and widely used. They are available in many forms, including liquid forms. Some liquid forms contain menthol as an antitussive. The menthol may be used in combination with other active pharmaceutical ingredients or alone. Most liquid cough suppressants, which do include menthol, use solvents such as ethyl alcohol or propylene glycol with surfactants to keep the menthol in solution.

Thus, a liquid antitussive cough suppressant medication that provides relief from coughing and soothes an irritated throat is desirable.

### SUMMARY OF THE INVENTION

The present invention is directed to a liquid antitussive composition having about 0.05 grams to about 0.2 grams of menthol per 100 ml of the liquid antitussive composition, a solvent system, and a surfactant, wherein the composition is stable for at least about 6 months at 40° C. and 75% relative humidity conditions.

The present invention includes a microemulsion antitussive composition comprising about 0.05 grams to about 0.2 grams of menthol per 100 ml of the antitussive composition, a solvent system, and a surfactant, wherein the composition is stable for at least about 6 months at 40° C. and 75% relative humidity conditions.

The present invention also includes a method of treating a pediatric subject for coughing comprising the step of administering to the pediatric subject, a liquid antitussive composition comprising about 0.05 grams to about 0.2 grams of menthol per 100 ml of the liquid antitussive composition, a solvent system, and a surfactant, wherein the composition is stable for at least about 6 months at 40° C. and 75% relative humidity conditions.

### DETAILED DESCRIPTION OF THE INVENTION

As used herein, “microemulsion” refers to a liquid mixture of oil, water and surfactant, which may optionally include a cosurfactant. A microemulsion is characterized by its clear, stable, and isotropic appearance.

As used herein, “stable” refers to a composition that is clear to the naked eye and substantially free from turbidity or oily globules. No phase separation should be observed in either aqueous and/or non-aqueous components for at least about 6 months at 40° C. and 75% relative humidity conditions. Additionally, the term “stable” means that at least about 90% of the originally added menthol is still present in the antitussive composition after about 6 months at 40° C. and 75% relative humidity conditions.

An unexpected feature of the inventive antitussive composition is the amount of menthol in the composition. Surprisingly, a stable liquid antitussive composition can be formulated with a menthol concentration of about 0.05 gram to about 0.2 grams of menthol per 100 ml of the liquid antitussive composition, in a solvent system containing a maximum of 0.5 grams of alcohol per 100 ml of liquid antitussive composition. A clear appearance is considered highly desirable by consumers, and is difficult to achieve. In the past, antitussive compositions formulated with 5 mg of menthol per 5 ml of composition either (i) did not exhibit solution stability, e.g., the composition appeared cloudy, or (ii) included high levels of alcohol, e.g., 4 grams of alcohol per 100 ml of solution, to keep the menthol in solution, thus giving the composition a clear appearance. Unexpectedly, Applicant has found that it is possible to formulate an antitussive composition containing greater amounts of menthol in an antitussive composition, which exhibits a clear appearance, without including excessive amounts of alcohol.

In one embodiment, the present invention is directed to an antitussive composition that contains 5 mg of menthol USP per 5 ml of antitussive composition. It was unexpectedly discovered that a clear solution containing 5 mg of menthol USP per 5 ml of the liquid antitussive composition could be made by formulating an antitussive composition with about 0.1 gram of menthol per 100 ml of the liquid antitussive composition, a solvent system and a surfactant.

An essential ingredient in the inventive antitussive composition is menthol. Menthol is widely used in a number of products and provides certain therapeutic qualities. Because of its cooling effect, menthol is often used in products to relieve skin irritation, sore throat, or nasal congestion. It is also commonly used as an antiseptic, in inhalants and as an analgesic. Desirably, when consumed or placed in the mouth, menthol has a cooling effect that relieves irritation. Menthol also has a strong minty taste that consumers find acceptable.

Typically in prior art formulations where a clear composition is desired, the amount of menthol added to an antitussive composition is limited by the amount of alcohol, i.e., ethanol, that is added. The alcohol solubilizes the menthol and keeps it in solution, thus enabling the solution to have a clear appearance.

However, the inventive antitussive composition is formulated in such a manner that enables higher levels of menthol to be included with lower levels of alcohol, while still maintaining a clear appearance that the consumer finds desirable.

Typically, menthol is included in the inventive composition in an amount, based upon the total weight of the antitussive composition, from about 0.05 grams to about 0.2 grams per 100 ml of the antitussive composition. Preferably, from 0.075 grams to about 0.15 grams per 100 ml of the antitussive composition. More preferably, from about 0.09 grams to about 0.1 grams per 100 ml of the antitussive composition. In a preferred embodiment, the menthol is present in an amount of about 0.1 grams per 100 ml of the antitussive composition. Menthol is a low melting solid. When liquefied, it forms an oil, which is then required to be solubilized within water and other solvents. Being an oil, it cannot be a true solution, instead it forms a microemulsion which is clear to the naked eye but tiny globules of oil can be seen under high magnification. These globules stay well separated with the help of surfactants (miscellar effect) but can potentially come together and coalesce into larger globules when subjected to stress such as high temperature, inadequate surfactant, changes to stable environment etc.

The Antitussive composition of the present invention includes a solvent system. The solvent system includes a non-aqueous part (e.g., glycerin, sorbitol solution (70% w/w), polyethylene glycol, and/or propylene glycol). Propylene glycol, also known by the systematic name propane-1,2-diol, is an organic compound with the chemical formula C₃H₈O₂. Under standard conditions (temperature of 25° C. and pressure of 100 kPa), it is a colorless, odorless, viscous liquid.

Propylene glycol has many uses. For example, it can be used as a moisturizer in food and medicines, and as a solvent for food colorings and flavorings.

It is a colorless, nearly odorless, clear, viscous liquid with a faintly sweet taste, hygroscopic and miscible with water, acetone, and chloroform.

The non-aqueous components of the solvent system are included in the antitussive composition in an amount from about 45 grams to about 75 grams per 100 ml of the antitussive composition. Preferably, from about 55 grams to about 65 grams per 100 ml of the antitussive composition. More preferably, from about 58 grams to about 63 grams per 100 ml of the antitussive composition.

Another essential ingredient in the antitussive composition is water. Water is the solvent that fills the gap after all other components have been added. Water is also essential to keep components such as gum in solution as well as other water soluble components with the product.

The amount of water that is included varies because the water is added to bring the composition up to a desired volume amount.

The solvent system also includes a co-solvent. The co-solvent is typically ethanol. Ethanol has widespread use as a solvent of substances intended for human contact or consumption, including scents, flavorings, colorings, and medicines. In the antitussive composition, the amount of alcohol, i.e., ethanol is less than about 0.5 grams of alcohol per 100 ml of liquid antitussive composition. In one embodiment, the alcohol is less than about 0.4 grams of alcohol per 100 ml of liquid antitussive composition. In another embodiment, the alcohol is less than about 0.3 grams of alcohol per 100 ml of liquid antitussive composition. In yet another embodiment, the alcohol is less than about 0.2 grams of alcohol per 100 ml of liquid antitussive composition. In still yet another embodiment, it is less than about 0.5 grams of alcohol per 100 ml of liquid antitussive composition.

A surfactant is included in the antitussive composition. The surfactant facilitates the formation of a clear, stable liquid product that remains clear under accelerated temperature conditions, i.e., temperature of 40° C. at 75% RH for 3 months or more. Suitable surfactants include, for example, nonionic surfactants. In one embodiment, the surfactant is a nonionic surfactant that is a polyoxyethylene-polyoxypropylene block co-polymer with the general formula HO(C₂H₄O)ₐ(—C₃H₆O)_{b}(C₂H₄O)ₐH. An example of one such polyoxyethylene-polyoxypropylene block co-polymer is Poloxamer 407, which is a hydrophilic non-ionic surfactant of the more general class of copolymers known as poloxamers and has a HLB value of 22. Poloxamer 407 is a triblock copolymer consisting of a central hydrophobic block of polypropylene glycol flanked by two hydrophilic blocks of polyethylene glycol. The approximate length of the two PEG blocks is 101 repeat units, while the approximate length of the propylene glycol block is 56 repeat units. This particular compound is also known by the BASF trade name PLURONIC F127. It can also be found in some mouthwashes and is available in different grades which vary from liquids to solids. It is also used as an emulsifying agent, solubilizing agent, surfactant, and wetting agent for antibiotics.

In another embodiment, the nonionic surfactant is polyoxyl 40 hydrogenated castor oil. An example of such a surfactant is CREMOPHOR RH 40, which has an HLB value of 16 and is also helpful in solubilizing Menthol. CREMOPHOR RH 40 is also available from BASF. If desirable, more than one nonionic surfactant (e.g., a polyoxyethylene-polyoxypropylene block co-polymer with the general formula HO(C₂H₄O)ₐ(—C₃H₆O)_{b}(C₂H₄O)ₐH and a polyoxyl 40 hydrogenated castor oil) may be included in the liquid antitussive composition.

The surfactant is included in the composition in an amount from about 0.2 grams to about 0.9 grams per 100 ml of the liquid antitussive composition. Preferably, the surfactant is about 0.3 grams to about 0.8 grams, and more preferably, about 0.4 grams to about 0.75 grams per 100 ml of the antitussive composition. In one embodiment, the surfactant is about 0.625 grams per 100 ml of antitussive composition.

Optionally, a variety of ingredients may be included in the antitussive composition of the present invention.

Any coloring agent suitable for use in a food or pharmaceutical product may be used in the present invention. Typical coloring agents include, for example, azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Similarly, a flavor may be included in the antitussive composition. The amount of flavor added to the composition is dependent upon the desired taste characteristics.

The composition may contain other components, such as aromas, high intensity sweeteners such as sucralose, viscosity modifiers such as xanthan gum to develop the desired texture and consistency, preservatives such as sodium benzoate NF, buffers such as citric acid, or mixtures thereof.

The antitussive composition may be a liquid composition in the form of a microemulsion.

The present invention also includes a method of treating a pediatric subject for coughing. The method includes the step of administering to the pediatric subject, a liquid antitussive composition comprising about 0.05 grams to about 0.2 grams of menthol per 100 ml of the liquid antitussive composition, a solvent system, and a surfactant, wherein the composition is stable for at least about 6 months at 40° C. and 75% relative humidity conditions.

The antitussive composition of the present invention may be made by any method known to those skilled in the art so long as it results in the desired composition.

Suitable methods include, for example, combining each ingredient in a mixing kettle, where the ingredients may be added sequentially or in any manner so long as the intended result is achieved. Moreover, the mixing action should be sufficient to incorporate each ingredient into the composition.

The following example is provided to further illustrate the compositions and methods of the present invention. It should be understood that the present invention is not limited to the examples described.

### Example 1

| No. | % w/v | mg/5 mL | ITEM DESCRIPTION |
|---|---|---|---|
| 1 | 0.15 | 7.50 | Xanthan Gum NF |
| 2 | 20.00 | 1000.00 | Glycerin 99.7% |
| 3 | 0.60 | 30.00 | Citric Acid Anhydrous USP |
| 5 | 0.325 | 16.250 | Sodium Benzoate NF |
| 6 | 0.06 | 3.00 | Sucralose NF |
| 7 | 5.00 | 250.00 | Honey White |
| 8 | 0.625 | 31.25 | Poloxamer 407 |
| 9 | 20.00 | 1000.00 | Sorbitol Solution |
| 10 | 20.00 | 1000.00 | Polyethylene Glycol 1450 NF |
| 11 | 0.50 | 26.33 | Ethyl Alcohol (95%) |
| 12 | 0.10 | 5.00 | I-Menthol USP |
| 13 | 1.375 | 68.75 | Propylene Glycol USP |
| 14 | 0.06 | 3.00 | N&A Lemon 539691T (Firmenich) |
| 15 | 0.30 | 15.00 | Nat Honey WONF 539690C (Firmenich) |
| 16 | | Q.S | Purified Water USP |

The formulation above has been demonstrated to give an acceptable product while it complies with ADIs and is adequately preserved as per tests.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed:

1. A liquid antitussive composition comprising:
about 0.05 grams to about 0.2 grams of menthol per 100 ml of liquid antitussive composition;
a solvent system; and
a surfactant,
wherein the composition has a maximum alcohol content of about 0.5 grams per 100 ml of the liquid antitussive composition, and
wherein the composition is stable for about 6 months at 40° C., 75% relative humidity conditions.

2. The composition of claim 1, wherein the solvent system includes:
non-aqueous components selected from the group consisting of glycerin, sorbitol solution, polyethylene glycol, propylene glycol, and mixtures thereof;
a co-solvent; and
water.

3. The composition of claim 2, wherein the non-aqueous components of the solvent system are about 50 grams to about 70 grams per 100 ml of the liquid antitussive composition.

4. The composition of claim 2, wherein the co-solvent is ethanol.

5. The composition of claim 1, wherein the surfactant is a nonionic surfactant.

6. The composition of claim 5, wherein the nonionic surfactant is selected from the group consisting of a polyoxyethylene-polyoxypropylene block co-polymer, a polyoxyl 40 hydrogenated castor oil and mixtures thereof.

7. The composition of claim 6, wherein the nonionic surfactant is a polyoxyethylene-polyoxypropylene block co-polymer with the general formula:
HO(C₂H₄O)ₐ(—C₃H₆O)_{b}(C₂H₄O)ₐH.

8. The composition of claim 1, wherein the surfactant is present in an amount of from about 0.2 grams to about 0.9 grams per 100 ml of the liquid antitussive composition.

9. The composition of claim 1, further comprising an optional ingredient selected from the group consisting of xanthan gum, citric acid, sodium benzoate, sucralose, flavors and mixtures thereof.

10. The composition of claim 1, wherein the composition further comprises dextromethorphan.

11. The composition of claim 1, wherein the composition is a microemulsion.

12. The composition of claim 1, wherein the composition is clear to the naked eye and free from any turbidity or oily globules.

13. The composition of claim 1, wherein the composition is at least about 90% of the originally added menthol is still present in the antitussive composition after about 6 months at 40° C. and 75% relative humidity conditions.

14. A microemulsion antitussive composition comprising:
about 0.05 grams to about 0.2 grams of menthol per 100 ml of antitussive composition;
a solvent system; and
a surfactant,
wherein the composition has a maximum alcohol content of about 0.5 grams per 100 ml of the antitussive composition, and
wherein the composition is stable for about 6 months at 40° C., 75% relative humidity conditions.

15. A method of treating a pediatric subject for coughing comprising the step of: administering to the pediatric subject, a liquid antitussive composition comprising about 0.05 grams to about 0.2 grams of menthol per 100 ml of the liquid antitussive composition, a solvent system, and a surfactant, wherein the composition is stable for at least about 6 months at 40° C. and 75% relative humidity conditions.
